(19) United States
(12) Patent Application Publication
(10) Pub. No.: US 2026/0297490 A1
(43) Pub. Date: Oct. 1, 2026

(54) CENTRIFUGAL SCREENING DEVICE

(71) Applicant: Feng Chia University, Taichung City (TW)

(72) Inventors: Chih-Hsin Shih, Taichung City (TW); Chia-Tse Shih, Taichung City (TW)

(21) Appl. No.: 19/564,715

(22) Filed: Mar. 12, 2026

(30) Foreign Application Priority Data
Apr. 1, 2025 (TW) 114112615

(51) Int. Cl. C12M 1/26; C12M 1/00; C12M 3/00; C12M 3/06

(52) U.S. Cl. CPC C12M 33/10; C12M 23/16; C12M 23/34; C12M 23/44

(57) ABSTRACT

The present invention relates to a centrifugal screening device composed of two fluidly connected portions, namely an inner disk and an outer ring. The inner disk includes a tree-like branched structure capable of providing a concentration gradient for cell growth. The outer ring includes multiple sample chambers for placing cells and allows repeated quantitative dosing and evacuation of culture medium. The present invention distributes different reagents into multiple mixed concentrations with gradient distributions through the tree-like structure of the inner disk, thereby enabling drug concentration testing on cells. Furthermore, the concept of combining the inner disk and the outer ring is applied to interchangeable functional unit disks and integrated into an automated centrifugal screening platform. This system establishes an automated workflow and reduces the cost associated with conventional cell-drug screening.

### FIELD OF INVENTION

The present invention relates to a screening device. More particularly, the present invention relates to a centrifugal screening device used on a centrifugal platform for screening specific substances, such as reagents, agents, cells, or drugs.

### BACKGROUND OF THE INVENTION

Drug screening is a step in the modern drug development process for examining and obtaining compounds having specific physiological activities. Essentially, the process of drug screening involves conducting pharmacological activity experiments on compounds. For screening purposes, it is necessary to conduct comparative analyses of the physiological activities of different compounds. Therefore, the experimental scheme for drug screening must exhibit standardized and quantitative characteristics. However, in existing drug screening processes, separating and screening reagents at different concentration ratios is relatively time-consuming and labor-intensive.

### SUMMARY OF THE INVENTION

In order to replace conventional drug screening experiments that are time-consuming and labor-intensive, and to provide a more efficient and accurate screening platform, the present invention provides a centrifugal screening device comprising an inner disk and an outer ring that are fluidly connected, wherein:

the inner disk comprises a circular disk base and a concentration configuration platform protruding upwardly from the circular disk base;

the concentration configuration platform comprises a first reagent groove and a second reagent groove disposed at a central portion thereof, the first reagent groove and the second reagent groove being semicircular, recessed, and adjacent to each other;

a first tree-like network channel and a second tree-like network channel respectively extending outwardly from bottoms of the first reagent groove and the second reagent groove and being in fluid communication therewith;

an outer edge of the concentration configuration platform including a plurality of protruding platforms and a plurality of recessed platforms forming a gear-shaped raised structure;

wherein the first tree-like network channel and the second tree-like network channel comprise curved tree-structured microfluidic channels including a plurality of liquid mixing points in fluid communication with each other;

wherein the first tree-like network channel and the second tree-like network channel extend to an end of the concentration configuration platform and terminate in a plurality of liquid outlets, each liquid outlet corresponding to one of the protruding platforms;

the outer ring comprising a hollow annular structure including an inner disk mounting hole, an edge of the mounting hole being configured to correspond to the protruding platforms and recessed platforms of the concentration configuration platform so as to form a gear-shaped opening allowing the outer ring to be detachably mounted on and fixed with the inner disk;

the outer ring extending outwardly from the edge of the inner disk mounting hole and being in fluid communication with each other further comprising: a liquid receiving hole, a sample chamber, a metering flow channel, a siphon flow channel, a waste liquid guiding chamber, and a waste liquid chamber; and

wherein the liquid receiving hole is aligned with the liquid outlets of the concentration configuration platform and is in fluid communication with the sample chamber, the sample chamber being a receiving space having the metering flow channel disposed on a right side thereof and communicating with the waste liquid guiding chamber, and the siphon flow channel disposed at a lower left side thereof and communicating with the waste liquid chamber.

The outer edge of the concentration configuration platform comprises ten protruding platforms and ten recessed platforms.

The waste liquid guiding chamber comprises an arc-shaped chamber extending laterally. Each tree-like network channel has a cross-section between 0.1 mm and 0.9 mm. The receiving space is a circular receiving space.

The inner disk and the outer ring are assembled together and rotated on a centrifugal platform at different rotational directions and speeds.

During a wetting stage the inner disk is assembled with an empty outer ring, a wetting liquid is introduced into the first reagent groove and the second reagent groove, and the device is rotated so that the wetting liquid fills the first tree-like network channel and the second tree-like network channel and removes air bubbles therein.

During a replacement stage a first reagent and a second reagent are respectively introduced into the first reagent groove and the second reagent groove and the device is rotated such that the wetting liquid within the tree-like network channels is replaced by the first reagent and the second reagent, excess wetting liquid being collected in the waste liquid chamber.

During a sample loading stage the outer ring used in the wetting stage and replacement stage is removed and replaced with another outer ring containing a sample in the sample chamber, the first reagent and the second reagent are again introduced into the reagent grooves, and the device is rotated such that mixtures of the reagents having different concentrations flow into the sample chambers, excess mixed solution being directed into the waste liquid chamber through the metering flow channel.

During an incubation stage the outer ring used in the sample loading stage is removed and assembled with another inner disk and the device is placed in an incubator.

During a discharge stage the device is removed from the incubator and rotated on a centrifugal platform to discharge liquid.

The wetting liquid comprises ethanol. The first reagent and/or the second reagent comprise a drug solution and/or a cell culture medium mixture solution. The sample comprises a cell culture chip.

From the foregoing description, the present invention provides the following beneficial effects and advantages:
1. The present invention develops an automated microfluidic centrifugal screening device based on a centrifugal platform. One application thereof is cell drug screening, which can replace conventional drug screening experiments that are time-consuming and labor-intensive. The centrifugal screening device mainly consists of two parts: the inner disk and the outer ring. The inner disk includes a branched structure capable of providing a concentration gradient for cell growth, and the outer ring includes multiple sample chambers for placing cells and allows repeated quantitative dosing and evacuation of culture medium. The present invention enables rapid generation of concentration gradients and automation of cell culture operations, thereby providing an efficient and low-cost platform for drug screening.
2. The present invention develops a microfluidic centrifugal screening device capable of generating concentration gradients. Through the tree-like structure, two reagents are distributed into multiple mixed concentrations having gradient distributions for performing drug concentration testing on cells. The concept of combining the inner disk and the outer ring is applied to interchangeable functional unit disks and integrated into an automated centrifugal chromatography platform, thereby establishing an automated process and reducing the cost of cell drug screening.

### BRIEF DESCRIPTION OF THE DRAWINGS

The steps and the technical means adopted by the present invention to achieve the above and other objects can be best understood by referring to the following detailed description of the preferred embodiments and the accompanying drawings.

FIGS. 1 and 2 are a perspective view and a top view of a first preferred embodiment of the inner disk of the present invention.

FIG. 3 is a partially enlarged view of the first preferred embodiment of the inner disk of the present invention.

FIGS. 4 and 5 are a perspective view and a top view of a first preferred embodiment of the outer ring of the present invention.

FIG. 6 is a partially enlarged view of the first preferred embodiment of the outer ring of the present invention.

FIG. 7 is a schematic view illustrating assembly of the outer ring and the inner disk of the present invention.

FIG. 8 is a schematic view illustrating rotational speeds used in the preferred embodiment of the present invention.

FIG. 9 is a schematic flow diagram illustrating a usage process of the centrifugal screening device of the present invention.

### DETAILED DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts. It is not intended to limit the method by the exemplary embodiments described herein. In the following detailed description, for purposes of explanation, numerous specific details are set forth in order to attain a thorough understanding of the disclosed embodiments. It will be apparent, however, that one or more embodiments may be practiced without these specific details.

As used in the description herein and throughout the claims that follow, the meaning of “a”, “an”, and “the” may include reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to.

Flowcharts are used herein to illustrate operations that may be performed by the system according to various embodiments of the present invention. It should be understood that the operations described herein are not necessarily required to be performed in the specific order shown. Rather, the operations may be performed in a different order, in reverse order, or concurrently, depending on the particular implementation. Furthermore, one or more additional operations may be included, and one or more operations may be omitted, without departing from the spirit and scope of the present invention.

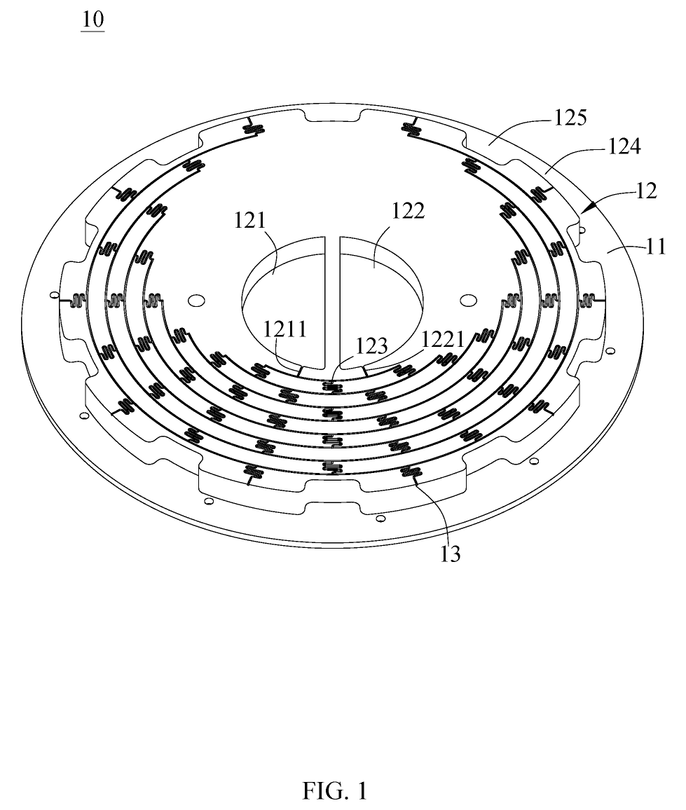

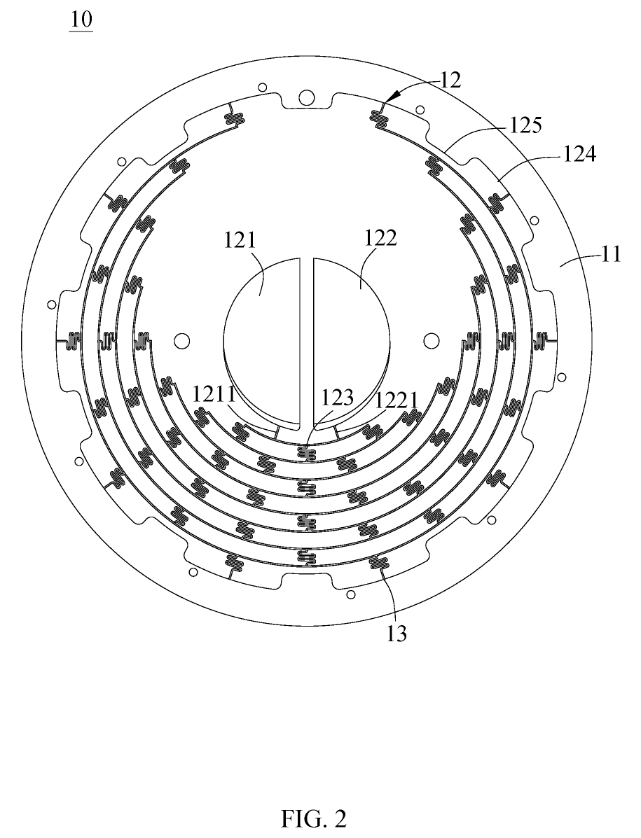

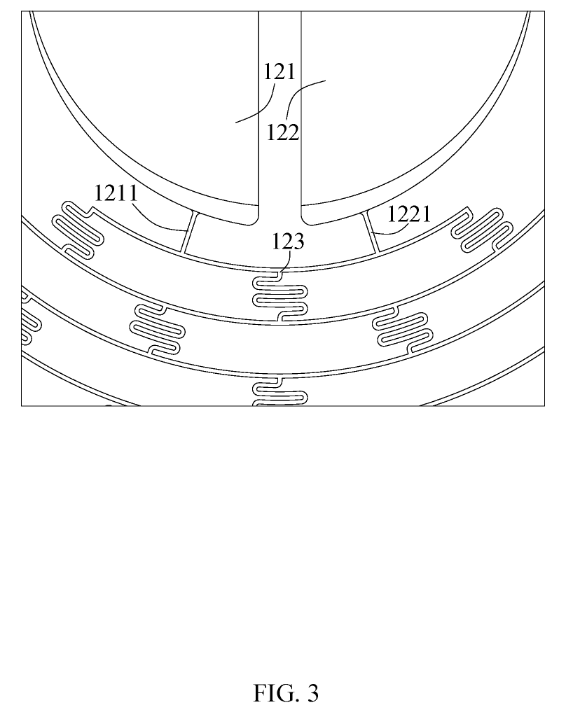

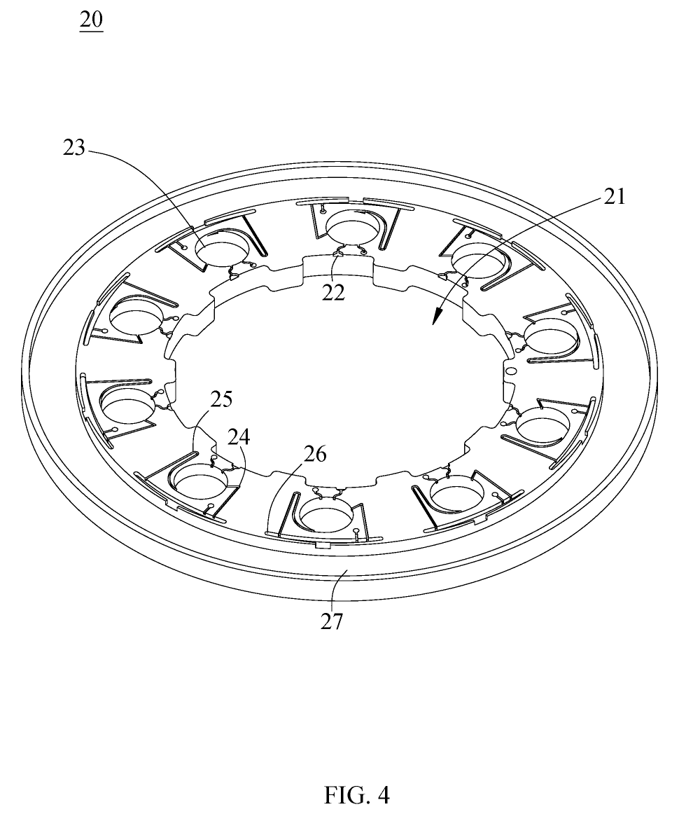

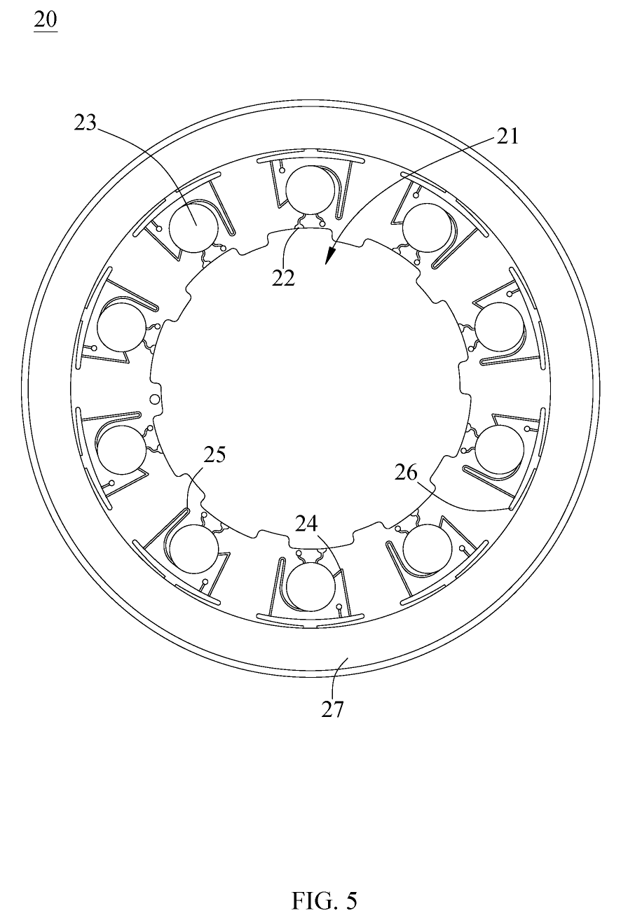

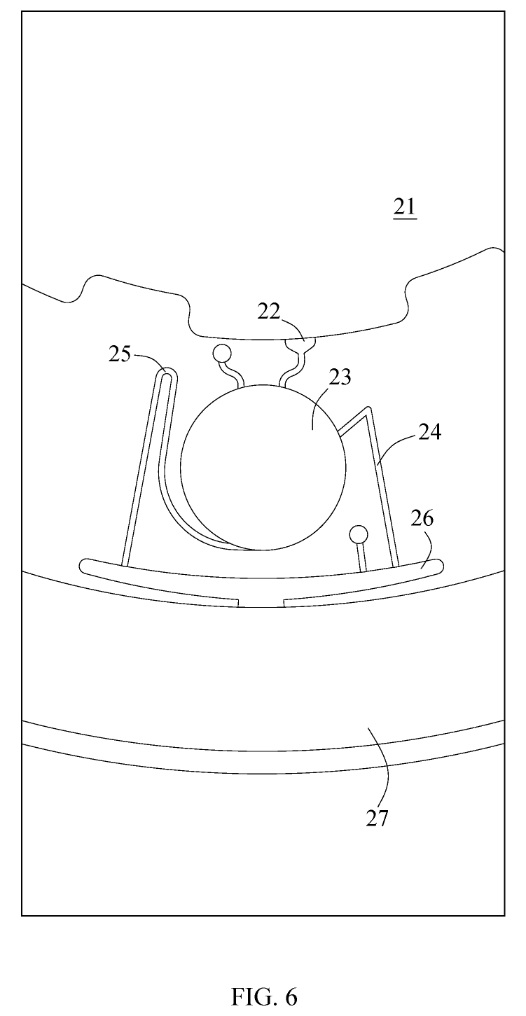

Please refer to FIGS. 1 to 7. The centrifugal screening device of the present invention comprises an inner disk 10 and an outer ring 20 that are in fluid communication.

### Inner Disk Embodiment 1

A first preferred embodiment of the inner disk 10 of the present invention is shown in FIGS. 1 to 3, and includes a circular disk-shaped base 11 and a circular upwardly protruding concentration configuration platform 12.

The concentration configuration platform 12 is provided at a central portion with a first reagent groove 121 and a second reagent groove 122 that are semicircular, adjacent, and recessed. A first tree-like network channel 1211 and a second tree-like network channel 1221 extend outwardly from the bottoms of the first reagent groove 121 and the second reagent groove 122, respectively, in fluid communication. An outer edge of the concentration configuration platform 12 includes a plurality of protruding platforms 124 and recessed platforms 125 forming a gear-shaped raised structure. In the preferred embodiment of FIG. 1, ten protruding platforms 124 and ten recessed platforms 125 are provided.

The first tree-like network channel 1211 and the second tree-like network channel 1221 are curved tree-structured microfluidic channels and include a plurality of liquid mixing points 123 that allow mutual fluid communication. A cross-section of each tree-like network channel is preferably between 0.1 mm and 0.9 mm, for example 0.2 mm.

The first tree-like network channel 1211 and the second tree-like network channel 1221 extend to the end of the concentration configuration platform 12 where a plurality of liquid outlets 13 are formed. Each liquid outlet 13 corresponds to one protruding platform 124 of the concentration configuration platform 12.

### Inner Disk Embodiment 2

A second preferred embodiment of the inner disk 10 of the present invention is substantially the same as the aforementioned first preferred embodiment, except that the microfluidic structures of the first tree-like network channel 1211 and the second tree-like network channel 1221 are not included.

### Outer Ring Embodiment 1

The outer ring 20 is a hollow annular structure. As shown in FIG. 1, the ring includes an inner disk mounting hole 21. An edge of the hole corresponds to the protruding platforms 124 and recessed platforms 125 at the outer edge of the concentration configuration platform 12 to form a gear-shaped opening, such that the outer ring 20 can be detachably sleeved onto and fixed with the inner disk 10. Structurally, the outer ring 20 includes, extending outward from the edge of the inner disk mounting hole 21, a liquid receiving hole 22, a sample chamber 23, a metering flow channel 24, a siphon flow channel 25, a waste liquid guiding chamber 26, and a waste liquid chamber 27 that are in fluid communication.

The liquid receiving hole 22 corresponds to the liquid outlets 13 at the end of the concentration configuration platform 12 for receiving liquid discharged from the liquid outlets 13 and communicates with the sample chamber 23. The sample chamber 23 is a receiving space, which in this embodiment is a circular receiving space. A metering flow channel 24 is provided on the right side of the sample chamber 23 and communicates with the waste liquid guiding chamber 26. A siphon flow channel 25 is provided at the lower left side of the sample chamber 23 and ultimately communicates with the waste liquid chamber 27.

The waste liquid guiding chamber 26 is an arc-shaped chamber extending left and right, capable of receiving liquid discharged from the metering flow channel 24 and the siphon flow channel 25 respectively, and guiding the liquid into the waste liquid chamber 27 through an arc-shaped flow path.

### Operation of the Centrifugal Screening Device

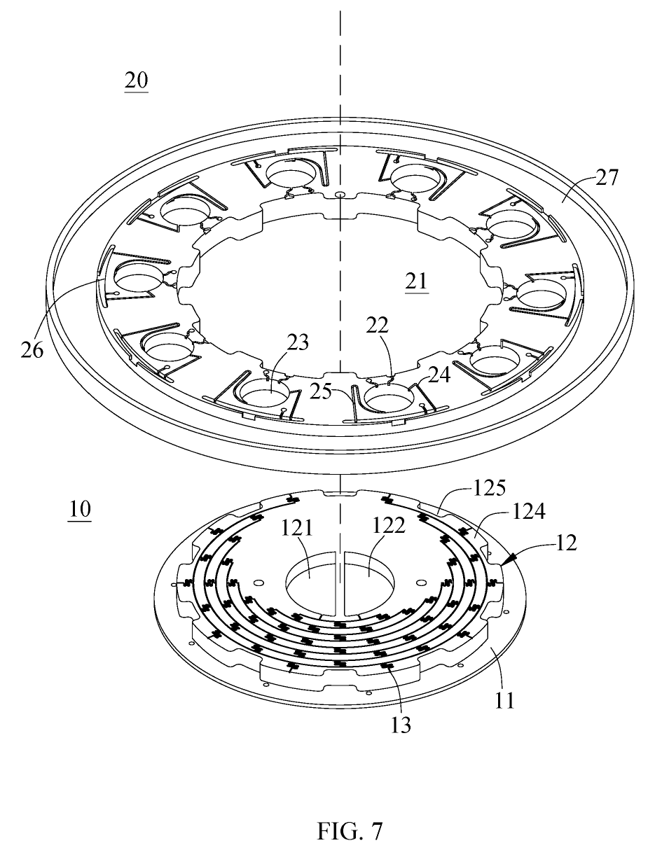

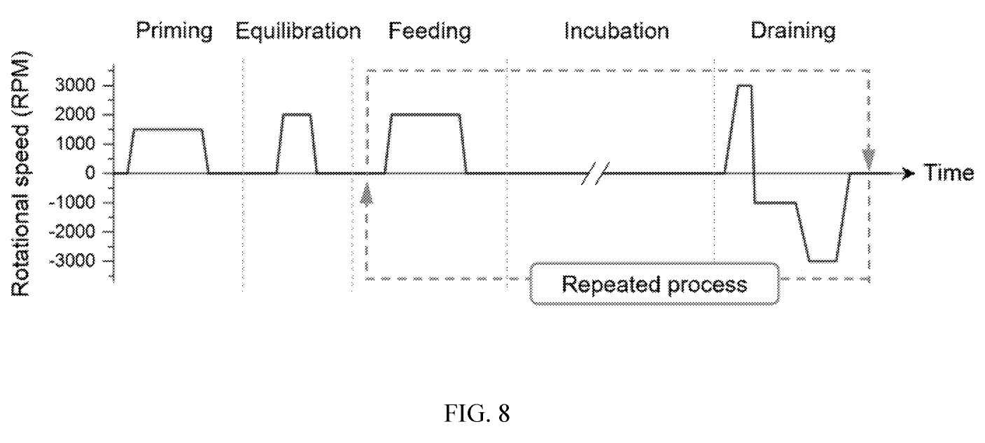

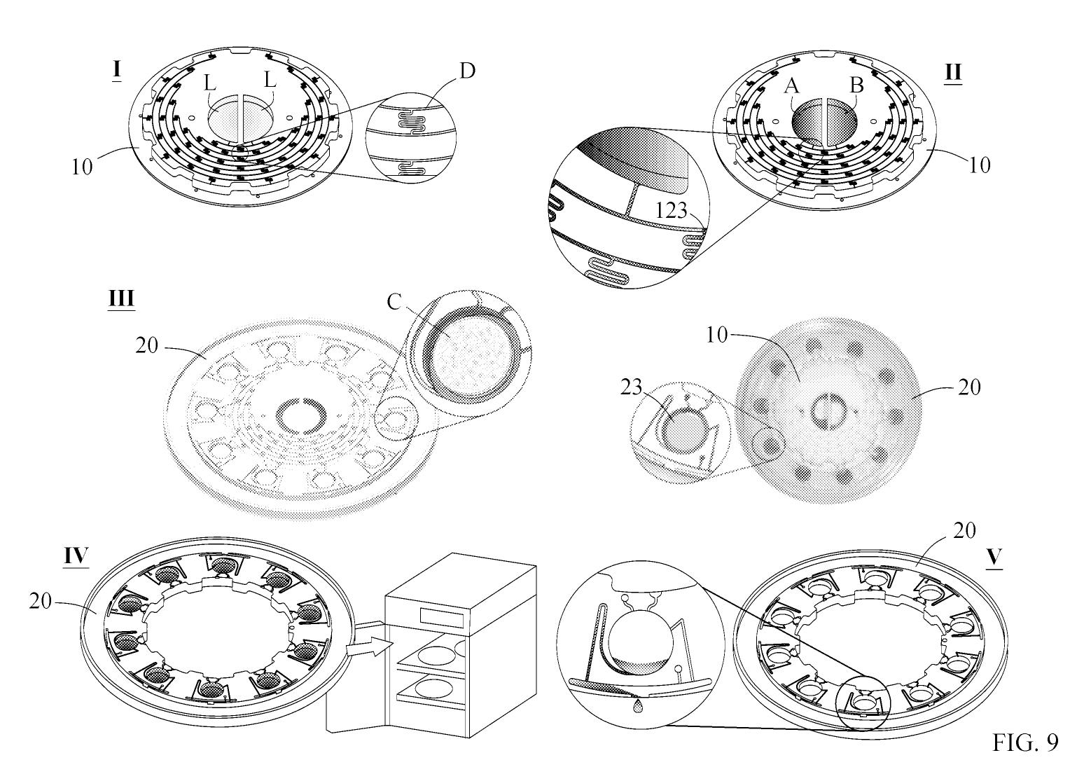

Please refer to FIGS. 7 to 9, in a preferred embodiment, the centrifugal screening device 100 is configured to function as a cell-drug screening platform. During operation, the inner disk 10 and the outer ring 20 are assembled together and placed on a centrifugal platform, such as a rotary centrifuge, where rotation is performed at different speeds. The operation preferably includes five stages: (I) wetting, (II) replacement, (III) sample loading, (IV) incubation, and (V) discharge.

Stage I Wetting Stage: During the wetting stage, the inner disk 10 of Embodiment 1 is assembled with an empty outer ring 20. A wetting liquid L having low surface tension and a low contact angle, such as ethanol, is introduced into the first reagent groove 121 and the second reagent groove 122 of the inner disk 10. The centrifugal screening device 100 is then rotated in a clockwise direction (CW) at approximately 1500 RPM. Due to the low surface tension and low contact angle between the wetting liquid L and the surface of the inner disk 10, the wetting liquid L is able to fill the first tree-like network channel 1211 and the second tree-like network channel 1221. During this process, air bubbles D formed within the curved tree-structured microfluidic channels are removed by the flow of the wetting liquid L.

Stage II Replacement Stage: Once the first tree-like network channel 1211 and the second tree-like network channel 1221 are filled with ethanol, a first reagent A and a second reagent B are introduced. The first reagent A may comprise, for example, a drug solution, and the second reagent B may comprise a cell-culture medium mixture solution. The first reagent A and the second reagent B are respectively introduced into the first reagent groove 121 and the second reagent groove 122. The centrifugal screening device 100 is then rotated clockwise (CW) at approximately 2000 RPM. Under centrifugal force, the wetting liquid L within the first tree-like network channel 1211 and the second tree-like network channel 1221 is displaced by the first reagent A and the second reagent B. Any excess wetting liquid L is collected in the waste liquid chamber 27 of the outer ring 20.

Stage III Sample Loading Stage: After completion of the wetting stage, the outer ring 20 used in Stage I is removed. A second outer ring 20 containing a cell culture chip C within the sample chamber 23 is assembled with the same inner disk 10 used in the previous stage. The first reagent A and the second reagent B are again introduced into the first reagent groove 121 and the second reagent groove 122. The centrifugal screening device 100 is then rotated clockwise at approximately 2000 RPM. Under centrifugal force, mixtures of the first reagent A and the second reagent B having different concentrations flow into the respective sample chambers 23. Excess mixed solution of reagents A and B is directed into the waste liquid chamber 27 through the metering flow channel 24, thereby enabling metering of the mixed solution volume within each sample chamber 23.

Stage IV Incubation Stage: After sample loading, the outer ring 20 used in Stage II is removed from the assembly. The outer ring 20 containing the cell culture chip C is then assembled with the inner disk 10 of Embodiment 2. The centrifugal screening device 100 is subsequently placed in an incubator for incubation. Because the inner disk 10 of Embodiment 2 does not include the microfluidic channel structure, the liquid receiving holes 22 of the outer ring 20 are effectively sealed during the incubation stage IV and the subsequent discharge stage V when liquid filling is not required. This configuration prevents leakage of liquid from the outer ring 20 during incubation. For repeated experiments, a new outer ring 20 containing a cell culture chip C may be assembled with the inner disk 10 of Embodiment 1 to repeat the sample loading process.

Stage V Discharge Stage: After incubation, the centrifugal screening device 100, consisting of the inner disk 10 of Embodiment 2 and the outer ring 20 containing the cell culture chip C, is removed from the incubator and placed on the centrifugal platform to perform a liquid discharge process. The rotational speed of the centrifugal screening device 100 is increased to approximately 3000 RPM in the clockwise direction. The rotational direction is then rapidly reversed to counterclockwise (CCW) and reduced to approximately 1000 RPM with an angular acceleration of approximately 50,000 RPM/s. This motion generates an Euler-force-assisted siphon effect, causing liquid within the sample chamber 23 to be discharged through the siphon flow channel 25 into the waste liquid chamber 27. Finally, the centrifugal screening device 100 is rotated at approximately 3000 RPM in the counterclockwise direction to remove residual liquid remaining in the siphon flow channel 25, thereby preventing unintended discharge during the next sample loading stage.

If the cell culture and drug testing processes continue, the centrifugal screening device 100 may repeat the drug concentration loading, incubation, and discharge stages.

Although the centrifugal screening device 100 is described above using a cell-drug screening platform as an exemplary embodiment, the first reagent A and the second reagent B are described as a drug solution and a cell-culture medium mixture solution respectively. However, the centrifugal screening device 100 of the present invention is capable of generating mixtures with various concentration gradients through mixing of the first reagent A and the second reagent B. Accordingly, the device may be applied to a wide variety of chemical, biological, medical, biomedical, or biochemical applications and is not limited to the specific embodiments described herein.

Flowcharts are used herein to illustrate operations that may be performed by the system according to various embodiments of the present invention. It should be understood that the operations described herein are not necessarily required to be performed in the specific order shown. Rather, the operations may be performed in a different order, in reverse order, or concurrently, depending on the particular implementation. Furthermore, one or more additional operations may be included, and one or more operations may be omitted, without departing from the spirit and scope of the present invention.

In certain embodiments, numerical values are used to describe quantities of components and properties. It should be understood that such numerical values used in describing the embodiments may, in some examples, be modified by terms such as “about,” “approximately,” or “substantially.” Unless otherwise specified, the terms “about,” “approximately,” or “substantially” indicate that the stated numerical values may vary by ±20%. Accordingly, in certain embodiments, numerical parameters set forth in the specification and the claims are approximate values that may vary depending upon the desired characteristics of individual embodiments. In some embodiments, such numerical parameters should be interpreted in view of the specified significant digits and in accordance with ordinary rounding practices. Although the numerical ranges and parameters used in certain embodiments of the present invention to define the scope of the invention are approximate values, in specific embodiments such numerical values are set as precisely as practicable within feasible limits.

Finally, it should be understood that the embodiments described herein are provided solely for the purpose of illustrating the principles of the embodiments of the present invention. Other variations and modifications may also fall within the scope of the present invention. Accordingly, alternative configurations of the embodiments of the present invention may be implemented in accordance with the teachings of the present invention as illustrative examples rather than limitations. Therefore, the embodiments of the present invention are not limited to the embodiments explicitly described and illustrated herein.

What is claimed is:

1. A centrifugal screening device comprising an inner disk and an outer ring in fluid communication, wherein:
the inner disk comprises a circular disk base and a concentration configuration platform protruding upwardly from the circular disk base;
the concentration configuration platform comprises a first reagent groove and a second reagent groove disposed at a central portion thereof, the first reagent groove and the second reagent groove being semicircular, recessed, and adjacent to each other;
a first tree-like network channel and a second tree-like network channel respectively extending outwardly from bottoms of the first reagent groove and the second reagent groove and being in fluid communication therewith;
an outer edge of the concentration configuration platform including a plurality of protruding platforms and a plurality of recessed platforms forming a gear-shaped raised structure;
wherein the first tree-like network channel and the second tree-like network channel comprise curved tree-structured microfluidic channels including a plurality of liquid mixing points in fluid communication with each other;
wherein the first tree-like network channel and the second tree-like network channel extend to an end of the concentration configuration platform and terminate in a plurality of liquid outlets, each liquid outlet corresponding to one of the protruding platforms;
the outer ring comprising a hollow annular structure including an inner disk mounting hole, an edge of the mounting hole being configured to correspond to the protruding platforms and recessed platforms of the concentration configuration platform so as to form a gear-shaped opening allowing the outer ring to be detachably mounted on and fixed with the inner disk;
the outer ring extending outwardly from the edge of the inner disk mounting hole and being in fluid communication with each other further comprising: a liquid receiving hole, a sample chamber, a metering flow channel, a siphon flow channel, a waste liquid guiding chamber, and a waste liquid chamber; and
wherein the liquid receiving hole is aligned with the liquid outlets of the concentration configuration platform and is in fluid communication with the sample chamber, the sample chamber being a receiving space having the metering flow channel disposed on a right side thereof and communicating with the waste liquid guiding chamber, and the siphon flow channel disposed at a lower left side thereof and communicating with the waste liquid chamber.

2. The centrifugal screening device according to claim 1, wherein the outer edge of the concentration configuration platform comprises ten protruding platforms and ten recessed platforms.

3. The centrifugal screening device according to claim 1, wherein:
the waste liquid guiding chamber comprises an arc-shaped chamber extending laterally;
each tree-like network channel has a cross-section between 0.1 mm and 0.9 mm; and
the receiving space is a circular receiving space.

4. The centrifugal screening device according to claim 1, wherein the inner disk and the outer ring are assembled together and rotated on a centrifugal platform at different rotational directions and speeds.

5. The centrifugal screening device according to claim 1, wherein during a wetting stage the inner disk is assembled with an empty outer ring, a wetting liquid is introduced into the first reagent groove and the second reagent groove, and the device is rotated so that the wetting liquid fills the first tree-like network channel and the second tree-like network channel and removes air bubbles therein.

6. The centrifugal screening device according to claim 5, wherein during a replacement stage a first reagent and a second reagent are respectively introduced into the first reagent groove and the second reagent groove and the device is rotated such that the wetting liquid within the tree-like network channels is replaced by the first reagent and the second reagent, excess wetting liquid being collected in the waste liquid chamber.

7. The centrifugal screening device according to claim 6, wherein during a sample loading stage the outer ring used in the wetting stage and replacement stage is removed and replaced with another outer ring containing a sample in the sample chamber, the first reagent and the second reagent are again introduced into the reagent grooves, and the device is rotated such that mixtures of the reagents having different concentrations flow into the sample chambers, excess mixed solution being directed into the waste liquid chamber through the metering flow channel.

8. The centrifugal screening device according to claim 7, wherein during an incubation stage the outer ring used in the sample loading stage is removed and assembled with another inner disk and the device is placed in an incubator.

9. The centrifugal screening device according to claim 8, wherein during a discharge stage the device is removed from the incubator and rotated on a centrifugal platform to discharge liquid.

10. The centrifugal screening device according to claim 6, wherein:
the wetting liquid comprises ethanol;
the first reagent and/or the second reagent comprise a drug solution and/or a cell culture medium mixture solution; and
the sample comprises a cell culture chip.

11. The centrifugal screening device according to claim 7, wherein:
the wetting liquid comprises ethanol;
the first reagent and/or the second reagent comprise a drug solution and/or a cell culture medium mixture solution; and
the sample comprises a cell culture chip.

12. The centrifugal screening device according to claim 8, wherein:
the wetting liquid comprises ethanol;
the first reagent and/or the second reagent comprise a drug solution and/or a cell culture medium mixture solution; and
the sample comprises a cell culture chip.

13. The centrifugal screening device according to claim 9, wherein:
the wetting liquid comprises ethanol;
the first reagent and/or the second reagent comprise a drug solution and/or a cell culture medium mixture solution; and
the sample comprises a cell culture chip.
